(19) United States
(12) Patent Application Publication
(10) Pub. No.: US 2007/0071682 A1
(43) Pub. Date: Mar. 29, 2007

(54) Methods for testing vaccine candidates against bacterial infection in rodents

(76) Inventors: John Fitzgerald Kokai-Kun, Frederick, MD (US); James Jacob Mond, Silver Spring, MD (US)

Correspondence Address: LAHIVE & COCKFIELD, LLP, ONE POST OFFICE SQUARE, BOSTON, MA, 02109-2127, US

(21) Appl. No.: 11/508,608

(22) Filed: Aug. 22, 2006

Related U.S. Application Data
(60) Provisional application No. 60/709,822, filed on Aug. 22, 2005.

(51) Int. Cl. A61K 49/00; A61K 39/12; A61K 39/02

(52) U.S. Cl. 424/9.2; 424/234.1; 424/204.1; 977/802

(57) ABSTRACT

Methods and a rodent model to test the effectiveness of vaccine candidates against bacteria, e.g., Staphylococcus aureus by systemically immunizing a rodent, particularly a cotton rat, with a vaccine candidate, intranasally challenging the cotton rat with the selected bacteria, and detecting a response with respect to an immune response, nasal colonization as a measure of the protective effect of the vaccine candidate, or both.

### RELATED APPLICATIONS

This application claims priority to U.S. Provisional Application No. 60/709822, filed Aug. 22, 2005, the entire contents of which are incorporated herein by reference.

### BACKGROUND OF THE INVENTION

Pathogenic bacteria, such as those from the genera Streptococcus, Pseudomonas, Micrococcus, Enterococcus, Corynebacterium, and Staphylococcus, account for a high amount of mortality and morbidity. Conventional treatments, such as antibiotic therapy, are sometimes, and are more readily becoming, ineffective against severe bacterial infections.

Staphylococcus aureus is typically regarded as the most pathogenic of the staphylococci. S. aureus infection remains one of the most common nosocomial and community-acquired infections. S. aureus is responsible for a wide range of infections, including soft tissue infections and potentially fatal bacteremias. The predominant treatment for such infections is the prescribed use of antibiotics. The effectiveness of antibiotic treatment, however, has declined in recent years, particularly with the continuing emergence of strains of S. aureus resistant to multiple antibiotics, such as methicillin-resistant S. aureus (MRS A), strains of S. aureus that are intermediately resistant to glycopeptides, and strains of S. aureus that are fully vancomycin resistant. As a result of these developments, S. aureus and other bacterial species threaten to become an even more difficult health problem to address, particularly in settings such as hospitals and nursing homes or with at-risk populations. Accordingly, with the potential threat of S. aureus nosocomial epidemics and the increasing need to limit the problems created by the use and misuse of antibiotics, alternative techniques to control S. aureus and other infection may be necessary.

The primary ecologic niche for S. aureus in humans is the nares, which are the source from which bacteria spread to other parts of the body. Approximately 20% of humans are persistently colonized intranasally by a single strain of S. aureus. Another 60% of individuals are intermittent nasal carriers of S. aureus strains that change with varying frequency. Only 20% are classified as persistent non-carriers. Generally, S. aureus colonization of the nares is asymptomatic, but the nasal carriage is a risk factor for staphylococcal infection, particularly in high risk populations.

Conventional systemic vaccine candidates have generally been evaluated on their induction of serum antibodies (e.g., immunoglobulin G (IgG)). Vaccine candidates systemically administered are not typically tested for their ability to induce a localized, secretory response (e.g., immunoglobulin A (IgA)) because no such response is typically expected.

To ensure compatibility, safety, and efficacy of potential treatments or prophylaxis in humans, researchers will likely rely on animal testing and models, as they have so routinely done in the past. Animal models for studying S. aureus and other bacterial infection and potential vaccine candidates have greatly contributed to the knowledge of virulence factors involved in disease. Conventional animal models for studying S. aureus and other bacterial infection, however, are limited in their applicability. Specifically, the development of agents or a combination of agents, such as vaccines (also interchangeably known herein as “agents” or “vaccine candidates” or “drug candidates”), for the prevention, management, treatment and/or inhibition of staphylococcal diseases is hindered by virulence of many bacterial species and strains. In addition, small animals used in such testing are relatively insensitive to a conventional, systemic challenge to induce a staphylococcal infection.

Generally, for example, a challenge amount of greater than about 5×10⁶ CFUs of S. aureus, or more for less virulent species or strains, is required to achieve reproducible infection in many small test animals. This minimum bacteria dose to achieve infection can easily overwhelm the immune response no matter how robust. This typically results in a lack of detectable protection against colonization or infection or even mortality in the subjects.

Thus, there is an unmet need in the art for methods of testing and evaluating proposed agents, such as vaccine candidates, against bacteria, such as S. aureus, that provide reproducible bacterial infection or colonization without challenge doses of bacteria that overwhelm the immune system of the animal subject so that detection of a protective immune response may occur. There is also an unmet need in the art for an animal model to evaluate the efficacy of a vaccine candidate to inhibit, manage, prevent, and treat bacterial infections or colonization, such as S. aureus nasal colonization. Specifically, the development of a useful rodent model would be of great benefit.

### SUMMARY OF THE INVENTION

The present invention relates to providing methods and models for evaluating agents, such as vaccine candidates, against bacteria, such as staphylococci, particularly S. aureus. Unexpectedly and surprisingly, in the present invention, a rodent candidate (interchangeably referred to herein as “rodent(s)”) can be immunized systemically and still be protected from nasal colonization despite the fact that a mucosally protective immune response was not expected via this route of immunization. Thus, the invention encompasses methods for testing vaccine candidates that provide protective efficacy against bacteria, such as S. aureus, by systemically immunizing (also generally known as

### DETAILED DESCRIPTION OF THE INVENTION

The present invention is directed to novel methods for testing and/or evaluating the potential of a systemically distributed agent or a combination of agents, preferably a vaccine candidate, to treat, prevent, inhibit, or manage a bacterial infection or colonization, such as bacteria from the staphylococcal genus, preferably S. aureus, in the mucosa of rodent candidates, such as the in the nares. The methods and models of the invention provide for the use of localized challenges of one or more bacterial species or strains for the evaluation of systemically administered agents, e.g., a vaccine candidate. Using such localized challenge (e.g., in the nares) the present invention can advantageously overcome the limitations of the conventional art by reducing the amount of bacterial challenge required to produce a reproducible colonization in the rodent candidate, and also minimizing or avoiding fatality due to overwhelming infection in the rodent candidate as often seen in systemic infection models. Thus the rodent candidate that produces an immune response to the bacterial challenge can be identified and suitable agents for preventing, treating, or managing bacterial infection or colonization can be selected.

The conventional art has been unable to consistently evaluate the effect of a systemically administered vaccine candidate, because of the difficulty or inability to produce reproducible infection, particularly of staphylococcal species such as S. aureus without a relatively high challenge dose of bacteria. Also, conventionally it has been necessary to administer an antibiotic, e.g., streptomycin, to clear out the normal nasal flora before the bacterial challenge to allow reproducible nasal colonization by an antibiotic resistant strain of S. aureus. In the present invention, however, any immune response that occurs is preferably detectable, e.g., there is a reduction or elimination of nasal colonization in the treated animals or there is prevention or inhibition of colonization. Preferably, the immunization and challenge can now occur and still provide a detectable response but without need for administering a preliminary or secondary agent, such as an antibiotic, to achieve reproducible colonization as these can cause complications or other adverse side effects and require additional time and effort in testing the agents for efficacy as well as requiring the use of antibiotic resistant strains of bacteria with which to conduct the challenge.

Conventionally, intranasal bacterial challenges would be useful only to test locally administered drugs or vaccines, which are administered locally in the nasal passages, to determine their effect on bacterial colonization based on the expected secretory IgA response. Localized bacterial challenge to a mucosal surface like the nares was believed to be inapplicable in evaluating the efficacy of a vaccine candidate administered systemically. Convention has held that a systemic immune response, e.g., via IgG, is not the same as a localized response, e.g., via IgA, and thus, would not be expected to be protective against colonization or infection by a bacteria on a mucosal surface. In some embodiments, the present invention relies on the systemic administration of a vaccine candidate to provide a surprising and unexpected local response that can be used to determine protective efficacy relative to intranasal bacterial challenge.

Without being bound by theory, it is believed that the morbidity or prematurely induced mortality in many rodent test subjects using conventional models is due to the requirement for large challenge doses of bacteria in many rodent models as compared to challenges of bacteria that would be expected to lead to infection in humans. Such doses may overwhelm any immune response and result in an erroneous clinical conclusion that the vaccine candidate was not effective for protection against the bacteria, such as S. aureus. Accordingly, the true efficacy of the vaccine candidate cannot be determined. Thus, typically it has been inconclusive whether certain vaccine candidates were effective in preventing, treating or managing an infection, or even whether the candidates initiated any or a sufficient amount of immune response to be protective.

Surprisingly, it has now been discovered that systemic vaccination using a vaccine candidate protective against a bacterial species or strain produces localized effects on mucosal surfaces, such as the nares, of a rodent subject, e.g., the cotton rat. The cotton rat model facilitates evaluation of vaccine candidate efficacy in treating or managing existing infection, or preventing or inhibiting future bacterial colonization in the nares of the rodent candidate. For example, in conventional rodent models involving an S. aureus challenge, about 5×10⁶ or greater S. aureus organisms must be systemically delivered to a rodent subject, such as a mouse or cotton rat, to produce a reproducible S. aureus infection. This amount of bacteria, however, typically overwhelms the rodent immune system and causes mortality (or morbidity), thereby preventing or rendering more difficult a determination as to whether the vaccine candidate had a protective immune system effect. In a preferred embodiment of the present rodent methods and model, lower doses of S. aureus may be used as a challenge to test the efficacy of vaccine candidates. Further, larger intranasal challenges with doses of S. aureus up to about 10⁹ bacteria are not fatal in rodents as long as they are administered in a volume that does not result in a substantial portion of the challenge reaching the lungs.

In one embodiment, the present invention is adaptable to bacterial species in which the amount of bacteria generally required to induce a reproducible infection in rodents is close to the amount of bacteria that is fatal to a significant portion or all of the rodent candidate population and higher than the amount of bacteria to cause infection in humans. For example, the methods and model of the present invention are adaptable to bacterial species that are members of the genera: Streptococcus, Pseudomonas, Micrococcus, Enterococcus, Corynebacterium, and Staphylococcus. This list of genera is not limited but is merely exemplary and preferred. The bacterial species or strain preferably used in accordance with the present methods and model includes one or more staphylococcal species. More preferably, the bacterial species to be tested includes S. aureus.

Methods that increase the likelihood of a reproducible bacterial infection or colonization in a rodent candidate using a lower amount of bacteria to prevent overwhelming a potentially protective immune response and minimize or avoid mortality or morbidity compared to conventional methods, which typically lead to mortality or morbidity, can provide a more readily detectable protective immune response in rodent candidates that would not have previously been detected using conventional methods where some or all of the rodent candidates are morbidly or mortally affected by the high challenge dose of the bacterial species being tested, regardless of the robustness of the immune response to the candidate agent being testing. Thus, challenging the rodent candidates intranasally with lower challenge amounts, e.g., smaller than the conventional amount of bacteria used to systemically challenge rodents, can produce a more reproducible infection in rodent candidates and can therefore more readily permit detection of any protective immune response. In accordance with the present invention, methods of the present invention facilitate determination of whether a localized (e.g., intranasal) immune response in the rodents is attributable to the systemically administered vaccine candidate.

In particular, the present invention is directed to methods for testing or evaluating the potential of an agent or a combination of agents, preferably a vaccine candidate, to treat, prevent, inhibit, or manage an infection or colonization, such as a staphylococci infection or nasal colonization, preferably an S. aureus infection or nasal colonization, in rodents. Surprisingly and unexpectedly, intranasal challenge, which can require less bacteria than systemic challenges to achieve reproducible results, and evaluation of intranasal bacterial colonization creates a useful method and model of determining the immune effect in rodents of vaccine candidates in one embodiment—even after systemic administration of the agent being tested.

Intriguingly, it has now been determined that systemic administration of the vaccine candidate may provide a detectable immune response in, and preferably provide protection to, the mucosal surfaces of the nares. Thus, researchers may now consistently test and evaluate the efficacy of vaccine candidates in the rodent by investigating the amount or extent of colonization of S. aureus in the mucosa of the rodent even after only a systemic immunization with the agent to be tested for response.

In accordance with the present invention, the rodent candidate receiving a systemic immunization and local challenge can produce a detectable immune response and exhibit protection against S. aureus, or other bacterial infection or colonization, if administered an effective amount of an agent against that type of bacteria. Thus, one “inoculating” or “vaccinating”) a healthy rodent candidate, particularly a cotton rat, with at least one vaccine candidate, and then intranasally challenging the rodent candidate with a sufficient amount of bacteria, preferably one or more staphylococcal species or strains such as S. aureus, to achieve a reproducible colonization. This amount of bacteria does not overwhelm the immune response of the rodent, thus allowing assessment of a protective immune response. In a preferred embodiment, the systemic immunizing is achieved through subcutaneous, intraperitoneal, transcutaneous or intramuscular administration of the vaccine candidate.

The invention also relates to rodent models for evaluating the efficacy of a vaccine candidate protective against bacteria, such as S. aureus, in a rodent candidate, by systemically immunizing the rodent candidate with a vaccine candidate, detecting a response, and intranasally challenging the rodent candidate with bacteria that colonizes the nares without overwhelming the immune response in the rodent candidate.

The invention also encompasses methods for screening a vaccine or a drug candidate for therapeutic efficacy against an infection by systemically immunizing a plurality of rodent candidates with the drug candidate, and intranasally challenging each rodent candidate with a sufficient amount of bacteria, such as S. aureus, to achieve a detectable nasal colonization in control non-immunized animals, e.g., in more than 50% of the control rodent subject population when a plurality of rodents are used, and determining the potential efficacy of the vaccine or drug candidate for the prevention, treatment, or management of the colonization based on the detectable response of the rodent candidate(s) to the vaccine or drug candidate.

Any of the embodiments described above or below stand independently although features may be combined to achieve preferred embodiments. Additional advantages and embodiments of the invention will also become more apparent to those of ordinary skill in the art upon review of the teachings of the present application.

### BRIEF DESCRIPTION OF THE DRAWINGS

Further features and advantages of the invention can be ascertained from the following detailed description that is provided in connection with the drawing(s) described below:

FIG. 1 illustrates results of Example 5, in which immunization of cotton rats with one purified protein antigen that protects the animals from nasal colonization and a second immunogen that is not protective, in accordance with one embodiment of the present invention.

embodiment of the present invention provides a model and methods in which intranasal challenge with S. aureus still permits the testing and evaluation of systemic agents to determine if the agents possess a prophylactic, preventative, therapeutic, or otherwise beneficial effect, to the rodent candidate in spite of the systemic route of administration of the agent. In one embodiment, fewer S. aureus organisms are administered in the challenge dose than in a traditional systemic challenge, e.g., fewer than 5×10⁶ organisms. Ultimately, the invention can also reduce the number of “false negatives” by promoting increased clinical accuracy in determining that certain vaccine candidates actually have protective immune response efficacy, which is facilitated by minimizing the challenge dose of bacteria to achieve reproducible colonization.

Importantly, the method of the present invention can now permit determination of the agent's role in protecting the rodent candidate against infection or colonization, or treating or managing infection or colonization, without factoring the confounding effect of whether the infection was simply a result of overwhelming (and irremediable) starting dose amount of bacteria, which normally causes rodent candidate mortality/morbidity no matter how robust the immune response induced by the agent. That is, the present invention can allow researchers to induce infection or colonization, such as S. aureus infection or nasal colonization, albeit locally (e.g., intranasally) of the rodent candidate to a level that facilitates accurate determination of the efficacy, if any, of a systemic vaccine candidate.

As used herein, rodent candidates (interchangeably referred to herein as “rats”) preferably refers to cotton rats (Sigmodon hispidus). Other animals suitable for use in the practice of this invention may include any other suitable rodent or rodent-like animal, including, but not limited to, hamsters, guinea pigs, chinchilla, mice and other types of rats. In the preferred embodiment, the rodent candidates are adults with fully functioning and healthy immune systems, able to produce both systemic and localized immune responses.

Embodiments of the present invention involve systemically administering to (e.g., immunizing and inoculating) the rodent candidate an agent, e.g., a vaccine candidate, a drug candidate, or a therapeutic composition, e.g., a monoclonal or polyclonal antibody preparation to be tested and evaluated for efficacy related to the prevention, treatment, or management of a bacterial infection or colonization. The rodent candidate is intranasally challenged with bacteria, e.g., S. aureus. Preferably, this challenge amount is less than what would conventionally be systemically administered to the rodent to provide a reproducible infection (generally known herein as the “systemic amount”) but sufficient to induce reproducible colonization of the nares in control untreated animals. The systemic amount is preferably selected to minimize or avoid inducing the immediate death of the rodent candidate. The immunizing and challenge may be in any order, and may be concurrent or sequential. Preferably, however, the immunizing of the agent into the rodent species occurs first, followed by challenge with the bacterial specie(s) against which an immune response is to be detected. For example, where the agent is administered after the challenge, it is typically a therapeutic agent being tested rather than a prophylactic one, e.g., a vaccine candidate. Preferably, the challenge occurs after administration of the agent.

In preferred embodiments of the present invention, the methods generally involve administering to a healthy rodent having a normally-functioning immune system one or more agents with the potential to prevent, treat, or manage a S. aureus infection or colonization. As used herein, an “agent,” a “vaccine candidate,” a “drug candidate,” or a “therapeutic composition” may be antibody-inducing antigens, one or more whole bacterial organisms or a population thereof, or a passively administered antibody preparation against an antigen, which includes surface antigens, virulence antigens, and adherence antigens, or any combination thereof. Surface antigens are typically accessible to an antibody when the antigen is in the configuration of the whole intact bacterium. Virulence antigens are typically antigens that are involved in the pathogenic process and are responsible for causing the disease. Adherence antigens typically mediate the ability of a staphylococcal bacterium to adhere to epithelial surfaces. Preferred vaccine candidates include one or more of a protein, carbohydrate, lipid, conjugate vaccine, antigenic peptide, DNA vaccines, viral expression vectors or any other macromolecule to which an antibody response can be induced.

Administration of the agent is preferably systemic. The agent is typically administered orally, rectally, parenterally (subcutaneously, intramuscularly, and intravenously), or transdermally. Subcutaneously, intramuscularly or transcutaneously are preferred. Indeed, the preferred method of administration is parenterally, specifically subcutaneously. Administration of the agent to be tested is not limited to these methods, and may include any suitable form of administration available to those of ordinary skill in the art that can consistently deliver the agent systemically to the rodent candidate. In some embodiments, the administration is in a manner which elicits at least a serum IgG response.

Dosage forms may include tablets, troches, dispersions, suspensions, absorptions, solutions, syrups, elixirs, capsules, patches, and the like. In addition to the common dosage forms set out above, the compounds of the present invention may also be administered by or in conjunction with controlled release means and/or delivery devices such as those described in, e.g., U.S. Pat. Nos. 3,845,770; 3,916,899; 3,536,809; 3,598,123; and 4,008,719, the entire disclosures of which are hereby incorporated by reference.

While the suitable amount of agent to be tested for its ability to provide a detectable and protective immune response will differ from agent to agent, (particularly depending on the particular bacterial species, the amount of bacterial challenge, the rodent species, and individual rodent subject features, such as weight, age, and immune system responsiveness) the invention facilitates determination of the effective amount of any particular agent against a particular type and amount of bacteria. In one variation, the amount of agent is sufficient to elicit an immune response against the bacteria, such as staphylococci, in normal, healthy rodent candidates having healthy immune systems. Generally, the amount of agent administered is about 0.1 μg to 250 μg per rodent candidate. Preferably, the amount of agent administered can be about 0.5 μg to 50 μg per rodent candidate. More preferably, the amount of agent administered can be about 1 μg to 10 μg per rodent candidate. The antigen may also be mixed with or formulated with or absorbed to and adjuvant, e.g., alum, or a combination of adjuvents.

Alternatively, other variations of the present invention involve a passive antibody administration, which may include monoclonal or polyclonal preparations. Preferably, the amount of agent administered is about 50 μg to 1000 μg per rodent candidate. More preferably, the amount of agent administered can be about 100 μg to 750 μg per rodent candidate. Even more preferably, the amount of agent administered can be about 250 μg to 500 μg per rodent candidate. The passive antibody preparation may also be administered by body weight of the rodent, e.g., 1-1000 mg/kg, more preferably 5-100 mg/kg, and even more preferably 10-50 mg/kg. In some variations of the present invention, the agent is administered once, while in other variations the agent is administered two, three, or four total times over the course of eight weeks, or, in the case of passive antibody administration, over each of one to four days.

Following administration of a final dose (or the single dose) of the agent to the rodent candidate, a latency period can be provided prior to the bacterial challenge. The duration of this period ranges from promptly or immediately in succession to about twelve (12) months or more, but preferably the period is about seven (7) days to about six (6) months after the administration of the agent. Preferably, the duration of this period ranges from about fourteen (14) days to about forty-two (42) days. In another preferred embodiment, the duration is from about seven (7) to twenty eight (28) days, preferably from about seven (7) to fourteen (14) days. In some embodiments, the duration of latency period lasts from the period immediately following administration of the agent to the time a host immune response is detected. In variations, the host immune response is determined by the detection of specific IgG response in the serum.

In some embodiments, the invention uses passive administration of antibodies. The duration of this period can be immediately after the final dose of antibody to six (6) months or more, after the administration of the antibodies. Preferably, the duration of this period ranges from about twelve (12) hours to about forty-eight (48) hours.

Preferably, the bacterial challenge is an amount of staphylococci (e.g., S. aureus) or other bacteria administered to the rodent candidate in one or more doses as part of the challenge. In accordance with the present invention, the bacterial challenge is administered locally to one or more mucosal surfaces or regions of the host, e.g., the rodent candidate. As used herein, the “nares” includes the nostrils, nasal passages, and areas adjacent thereto, such as mucosal surfaces. In the preferred embodiment, the bacterial challenge is administered intranasally.

A preferred dosage form for the bacterial challenge is in the form of a bacterial suspension, such as a S. aureus suspension. A suspension can be based on any suitable suspension for the challenge bacterial species. Preferably, the suspension includes a phosphate buffer saline (PBS) with a preferred suspension equivalent to about 10⁴ to 10⁷ CFU/mL. An exemplary percent transmittance is about 10%, which is approximately equivalent to about 10⁹ CFU/mL. The concentration of suspended S. aureus (or other bacterial specie(s)) can range from about 10³ CFU to about 10¹¹ CFU per animal, preferably from about 10⁴ CFU to about 10⁷ CFU per animal, and more preferably from about 10⁵ CFU to about 10⁶ CFU per animal.

The intranasal challenge volume or suspended bacteria per animal can range from about 1 μL to about 100 μL, preferably from about 5 μL to about 50 μL, and more preferably from about 10 μL to about 20 μL. The challenge dose may be prepared in any suitable manner available to those of ordinary skill in the art. For example, strains of S. 5 aureus are grown overnight on Columbia agar supplemented with 2% NaCl. In some variations, the S. aureus is grown to induce capsule formation. The bacteria can be washed by suspension in PBS. The suspension can be pelleted by centrifugation and then resuspended in 10 μL of PBS per animal. A preferred suitable method is provided in Kokai-Kun et al., Lysostaphin Cream Eradicates Staphylococcus aureus Nasal Colonization in a Cotton Rat Model, Antimicrobial Agents and Chemotherapy, Vol. 47, No. 5, pages 1589-1597 (2003), which is incorporated herein by express reference thereto. The challenge dose is preferably administered, when in liquid form, in a drop-wise fashion distributed equally in each nostril of the animal as a single challenge dose. Other suitable dosing regimens may be readily envisioned and attained by one of ordinary skill in the art.

As used herein, a reproducible infection or colonization, e.g., S. aureus, is one in which the amount of bacterial challenge among a sample population of rodent candidates is sufficient to produce a consistent and reproducible clinical diagnosis of an intranasal species-specific colonization in the sample population of rodent candidates, as determined by one of ordinary skill in the art. That is, unlike conventional methods, which involve systemic bacterial challenges that often overwhelm the immune systems of the rodent candidates leading to morbidity or mortality following the bacterial challenge, embodiments of the present invention can preferably induce reproducible colonization, wherein the rodent candidates are challenged intranasally with a lower dose of staphylococci or other bacteria to allow researchers to evaluate the effect of potential agents administered systemically against the infection. Challenging intranasally with a lower dose of bacteria, e.g., one or more staphylococci strain or combination thereof, allows a potential immune response induced by a systemically administered vaccine candidate with an opportunity to protect the test subjects from the bacterial challenge. This method and model preferably use a cotton rat as the rodent candidate test subject.

Generally, the amount of bacteria administered to the subject is sufficient to produce colonization, preferably reproducible nasal colonization control untreated animals. In some embodiments, the “sufficient amount of bacteria in the bacterial challenge” is from a minimum amount of bacteria to elicit detectable nasal colonization in the rodent candidate up to a maximum amount of bacteria that is insufficient to overwhelm a potential protective immune response. In other embodiments, the challenge is a sufficient amount of bacteria, such as S. aureus, to achieve detectable nasal colonization, in more than 50% of the untreated control rodent population, preferably in 75% of the untreated control rodents and more preferably 90% of the untreated control rodents.

Preferably, the method of the present invention involves an intranasal bacterial challenge using fewer organisms than a corresponding conventional systemic challenge amount for the same species to provide a reproducible infection. For example, the conventional systemic bacterial challenge typically involves an amount greater than about 5×10⁶ for S. aureus. In one embodiment, the amount of S. aureus in the bacterial challenge according to the invention is from about 10³ to 10⁷, preferably from about 10⁴ to 10⁶. These ranges present particularly suitable amounts of S. aureus bacteria for the present methods, although to provide a reproducible colonization greater amounts will typically be required for strains that colonize less well and lesser amounts will be required for more better colonizing strains, as will be readily known or determined by those of ordinary skill in the art, particularly in view of the disclosure herein.

The invention encompasses a model for providing reproducible bacterial colonization in a rodent candidate. The model allows the introduction of an intranasal bacterial challenge without overwhelming the immune response in the rodent candidate. The model includes immunizing a rodent or population of rodent candidates with a vaccine candidate. The rodent candidate or population are then suitable for testing and evaluating of one or more vaccine candidates, e.g., anti-staphylococci candidates or candidates for testing other bacterial species or strains.

The present invention can be advantageously used, for example, to develop vaccines or to evaluate potential therapeutic agents for efficacy against staphylococcal or other bacterial infections or colonization. Using the rodent model of the present invention, a vaccine successfully protecting, preventing, treating, or managing bacterial infection or colonization in the rodent candidate population can be more readily discovered for use against infection or colonization by the challenge bacteria.

The efficacy of an agent against a particular type of bacteria or combination of types of bacteria can be assessed in individual rodent candidates (e.g., by clinical evaluation) or in a representative sampling (e.g., population or samples) of rodent candidates (e.g., by clinical evaluation of groups of treated versus untreated/control treated rodent candidates). Thus, the invention provides a method and model for testing the efficacy of an agent by comparing treated and untreated groups of rodent candidates and observing whether the treated group of rodent candidates (e.g., those receiving the agent) exhibits an improved clinical profile compared to the untreated (control) group, e.g., reduced or eliminated nasal colonization by the challenge bacteria.

The effectiveness of the agent as a vaccine or a therapy, or alternatively the resistance of an animal to infection, can be evaluated by any means that directly or indirectly measures one or more parameters or symptoms associated with an infection or colonization, such as the amount of bacterial nasal colonization at some point after challenge. Various methods are well known to those of ordinary skill in the art, and any of these detection methods or assays are acceptable according to the invention. For example, an agent's efficacy can be directly measured by detecting or determining the amount of bacterial colonization in the mucosa of the nares. One suitable method is provided in Kokai-Kun et al., Lysostaphin Cream Eradicates Staphylococcus aureus Nasal Colonization in a Cotton Rat Model, Antimicrobial Agents and Chemotherapy, Vol. 47, No. 5, pages 1589-1597 (2003), which is incorporated herein by express reference thereto. Other suitable methods are provided by Tortora et al., Microbiology: An Introduction, Fourth Edition, 1992, incorporated herein by reference.

In a preferred embodiment, the agent's efficacy may be evaluated by determining the reduction of bacterial colonization in mucosa, when appropriate, as determined by one of ordinary skill in the art, by comparing the amount of bacterial colonization in rodents immunized with a vaccine candidate versus the amount of bacterial colonization in control, unimmunized or sham immunized rodents. In one embodiment, the methods of the invention can be used to identify agents that result in a significant reduction in bacterial colonization in the nares as compared to an appropriate control. As used herein, a significant reduction in bacterial colonization is greater than or equal to 25%, preferably greater than or equal to 50%, more preferably greater than or equal to 75%, and even more preferably greater than or equal to 90% reduction in colonization. In some embodiments, the reduction in bacterial colonization is substantially complete and preferably is 100% eliminated, (e.g., the bacteria and its colonies have been eradicated as far as can be detected).

In another preferred embodiment, the agent's efficacy may be evaluated by determining the reduction of bacterial colonization in mucosa, when appropriate, as determined by one of ordinary skill in the art, by comparing the amount of bacterial colonization before administration of a therapeutic candidate and after administration of a therapeutic candidate. As used herein, a significant reduction in bacterial colonization is greater than or equal to 25%, preferably greater than or equal to 50%, more preferably greater than or equal to 75%, and even more preferably greater than or equal to 90%. In some embodiments the reduction in bacterial colonization is substantially complete and is 100% eliminated (e.g., the bacteria and its colonies have been eradicated as far as can be detected). In these preferred embodiments, other parameters of efficacy can also be tested besides reduction, e.g., inhibition or prevention of colonization compared to an unvaccinated control.

Preferably, the vaccine candidate induces host immunity in the rodent candidate. The host immunity can include producing a humoral or cell mediated response, or both. For example, serum IgG responsive to the vaccine candidate, producing secretory IgA responsive to the vaccine candidate, or both may be produced.

Generally, the time at which the agent is evaluated following the bacterial challenge is known in the art and will depend on the rodent candidate, the type of bacteria, and related factors, as is readily understood. Alternatively, those of ordinary skill in the art will be able to readily determine a suitable period following which the efficacy of the agent can be best evaluated. The time may be based on the type of bacteria used, wherein the bacteria is permitted to colonize in the subject. In other embodiments, a vaccine candidate responsive to the bacterial challenge is evaluated for its effectiveness from about 1 to 30 days after the challenge. Preferably, the evaluation is from about 3 to about 25 days after the challenge, more preferably from about 5 to about 15 days after the challenge, and even more preferably about 7 days after the challenge. In some embodiments, the cotton rat subjects are sacrificed, or a population is sacrificed at specified intervals, to obtain access to the colonization in the nares for detection, while in some embodiments, the cotton rat subjects are living and anaesthetized for detection.

The effectiveness of a vaccine candidate, in accordance with other embodiments of the present invention, may also be measured by detecting the immune response in the host. In one embodiment, a vaccine candidate is effective if IgG and/or IgA specific for the vaccine candidate are detected in serum and in the mucosa, respectively. In another embodiment, a vaccine candidate is effective if IgA is detected in the mucosal surfaces of the nares. In some embodiments, immune response in the host can be measured by determining the status of other immune system parameters well known to those of skill in the art (e.g., including, but not limited to, cytokine expression and activity, T cell activation, etc . . . ).

Any other suitable test available to those of ordinary skill in the art may be implemented to determine and/or detect the efficacy of a particular agent. Alternative parameters that can be used to evaluate the efficacy of an agent include mortality or morbidity or nasal colonization, so long as a statistically significant portion of the candidate population remains alive and a significant portion of the control animals are infected or colonized.

As used herein, the terms “efficacy,” “protective efficacy,” or “protective against” each encompass both partial and complete protection or efficacy, e.g., when compared to an appropriate control (such as an untreated animal).

The terms “a therapeutically effective amount,” “a prophylactically effective amount,” and “an effective amount,” as used herein, are encompassed by the above-described dosage amounts and dose frequency schedule, particularly when coupled with prevention, treatment, or management of one or more bacterial strains. Preferably, the vaccine candidates are administered in an effective amount according to the invention.

The term “preventing,” as used herein, also refers to inhibiting colonization by or growth of bacteria. The term “treating,” as used herein, also refers to reducing existing colonies or bacteria. The term “managing,” as used herein in connection with “preventing, treating, and managing” a particular bacterial challenge, also includes partial or complete prevention and treatment, as well as any beneficial modification of candidate status or the course of colonization or infection or symptoms or detection thereof.

In other embodiments, the term “substantially,” as used herein is intended to include variations from the absolute condition, e.g., about 90 percent, preferably about 95 percent, more preferably about 99 percent of the absolute condition. For example, the term “substantially” in reference to reduction of the bacterial colonization content refers to reducing at least about 90 percent of the total colonization in each rodent or the total number of rodents colonized in a group of like-treated rodents. In preferred embodiments, the term “substantially” refers to at least about 99.9 percent or at least about 99.99 percent of the absolute.

The term “about,” as used herein, should generally be understood to refer to both numbers in a range of numerals. Moreover, all numerical ranges herein should be understood to include each whole integer within the range.

### EXAMPLES

The following examples are not intended to limit the scope of the invention, but merely to illustrate representative possibilities concerning the present invention.

### Example 1

### Inoculation of a Rodent Candidate

A healthy rodent candidate (preferably a cotton rat) having a normally functioning immune system is administered an agent to be tested for immune specificity against a selected bacterial strain or plurality of strains, such as S. aureus. As used herein for the Examples, immune specificity means the agent provides some level of immunological protection against (e.g., treatment for, prevention of, or management of) infection or colonization by S. aureus, although other strains or bacteria may be selected. The rodent candidate(s), e.g., cotton rat(s), are injected subcutaneously with 3 to 10 μg of the agent administered with or without an appropriate adjuvant. Administration can occur once weekly or bi-weekly for up to five doses. A period of 14 days can be provided before intranasally challenging the rodent candidate with the S. aureus.

### Example 2

### Challenging the Rodent Candidate with S. aureus

Following immunization, the rodent candidate is then challenged with S. aureus. About 10⁵ of S. aureus are dripped intranasally to the nares of adjacent mucosal surfaces in the rodent candidate in a total volume of 10 μL.

### Example 3

### Testing of Vaccine Candidates in Cotton Rats

Groups of cotton rats (the rodent candidate selected as being exemplary for the Examples herein) are immunized subcutaneously with three doses of killed S. aureus in adjuvant, or with adjuvant alone, over six weeks. The rats are then challenged intranasally with S. aureus. Immunization with killed bacteria would desirably result in production of anti-S. aureus-specific IgG in the serum and possibly but less likely, IgA in the saliva, and it would ideally reduce nasal colonization in rats after bacterial challenge.

### Example 4

### Protection of Cotton Rats from S. aureus Nasal Colonization by Subcutaneous Immunization with UV-Killed S. aureus

Groups of female cotton rats were immunized with either Ribi adjuvant alone (control) of 10⁸ UV-killed capsular type 5 S. aureus, receiving doses on days 1, 14 and 28. On day 42, the rats were challenged with 6×10⁵ of a heterologous methicillin resistant S. aureus (MRSA) strain. The rats were sacrificed 7 days post challenge and nasal colonization was determined by plating surgically removed and bisected noses on selective agar. The results are shown in Table 1 and Table 2.

TABLE 1

| Immunogen | Animal | Challenge | Result |
|---|---|---|---|
| UV-killed | Cotton | 6 × 10⁵ MRSA | Cotton rats nasally colonized |
| S. aureus | rats | | Control: 5/5, Average: 2655 |
| type 5-10⁸ | | | CFUs/nose |
| 3 times of 6 | | | Vaccinated: 6/9, Average 170 |
| weeks | | | CFUs/nose |

TABLE 2
CFUs recovered per nose

| Rat Number | CFUs/nose |
|---|---|
| Control 1 | >10,000 |
| Control 2 | 440 |
| Control 3 | 1145 |
| Control 4 | 2050 |
| Control 5 | 385 |
| Control 6 | 1905 |
| Vaccinated 1 | 25 |
| Vaccinated 2 | 210 |
| Vaccinated 3 | 460 |
| Vaccinated 4 | 0 |
| Vaccinated 5 | 25 |
| Vaccinated 6 | 0 |
| Vaccinated 7 | 35 |
| Vaccinated 8 | 270 |
| Vaccinated 9 | 0 |

Immunization via subcutaneous injection with a heterologous strain of UV-killed S. aureus protected cotton rats from nasal colonization by a nasally instilled challenge of MRSA.

### Example 5

### Immunization of Cotton Rats with one Purified Protein Antigen Protects the Animals from Nasal Colonization with a Second Immunogen

Groups of 10 cotton rats were subcutaneously immunized in Ribi adjuvant with one of two purified protein antigens isolated from S. aureus, or sham immunized with adjuvant alone. These immunizations occurred on days 1, 14 and 28, and the rats were intranasally challenged with 4×10⁶ of an MRSA strain on day 42 following obtaining a serum sample to determine serum levels of IgG. The animals were sacrificed on day 7 after the challenge and nasal colonization was determined.

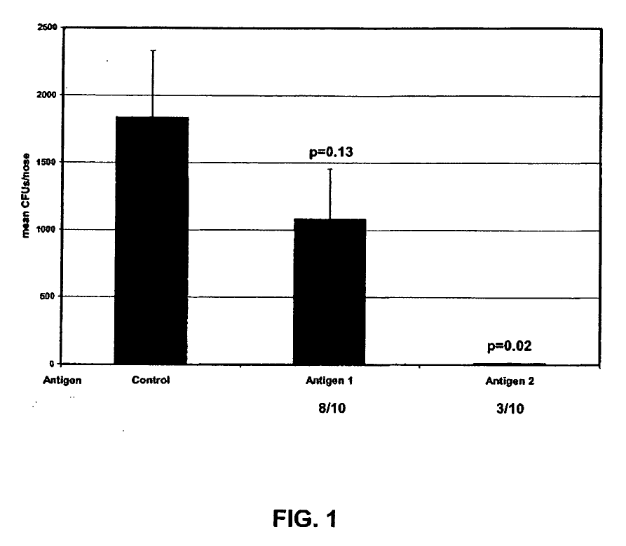

As shown in FIG. 1, only animals immunized with antigen 2 demonstrated a significant reduction in nasal colonization as compared to control animals. The average anti-antigen 1 titer in the antigen 1 group was 14.9 while the average anti-antigen 2 titer in antigen 2 group was 6.5. This example demonstrates that even though immunization with antigen 1 lead to a high titer serum antibody levels, this antibody response was not protective, while the lower serum response to antigen 2 was sufficient to protect the animals from nasal colonization by S. aureus. Thus, the rodent model of the invention is discriminatory and allows differentiation between an antibody response to a specific antigen which protects from nasal colonization and an antibody response that is not protective.

Although preferred embodiments of the invention have been described in the foregoing description, it will be understood that the invention is not limited to the specific embodiments disclosed herein but is capable of numerous modifications by one of ordinary skill in the art. It will be understood that the materials used and the chemical and biotechnological details may be slightly different or modified from the descriptions herein without departing from the methods and models disclosed and taught by the present invention.

### Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

What is claimed is:

1. A method for characterizing vaccine candidates protective against bacteria which comprises:
systemically immunizing a rodent candidate with at least one vaccine candidate;
challenging the rodent candidate in or adjacent to the nares with a sufficient number of bacteria to provide colonization of the bacteria in the nares of the candidate; and
characterizing an immune response in the rodent candidate to thereby characterize protective effects of vaccine candidates against bacteria.

2. The method of claim 1, wherein the bacteria comprises at least one organism of at least one genera selected from the group consisting of Streptococcus, Pseudomonas, Micrococcus, Enterococcus, Corynebacterium, and Staphylococcus or combinations thereof

3. The method of claim 1, wherein the rodent candidate is a cotton rat.

4. The method of claim 1, wherein the bacteria comprises a member of the genus Staphylococcus.

5. The method of claim 4, where in the bacteria comprises Staphylococcus aureus.

6. The method of claim 1, wherein the vaccine candidate is chosen from a population of bacterial cells, a population of killed bacterial cells, a protein, a group of proteins, a carbohydrate, a lipid, a conjugate vaccine, an antigenic peptide, a DNA vaccine, an antigen presented as part of a viral particle or any other macromolecule to which an antibody response can be induced.

7. The method of claim 6, wherein the vaccine candidate is mixed with an adjuvant or a combination of adjuvants.

8. The method of claim 1, wherein the systemic step of immunizing is via an oral route, a subcutaneous route, an intramuscular route, an intraperitoneal route, atranscutaneous route, via intravenous administration, or a combination thereof.

9. The method of claim 8, wherein the step of immunizing is via a subcutaneous route.

10. The method of claim 1, wherein the vaccine candidate induces host immunity in the rodent candidate.

11. The method of claim 10, wherein the host immunity includes producing serum IgG responsive to the vaccine candidate.

12. The method of claim 11, wherein the host immunity includes producing secretory IgA responsive to the vaccine candidate.

13. The method of claim 1, wherein the step of challenging comprises administering less than or equal to about 5×10⁹ of S. aureus.

14. The method of claim 1, wherein the step of challenging comprises administering 10³-10⁹ CFU of S. aureus.

15. The method of claim 5, wherein the amount of S. aureus is from about 5×10⁴ to 5 ×10⁶ CFU.

16. The method of claim 1, wherein the step of challenging comprises administering an amount of S. aureus sufficient to challenge the rodent candidate so as to provide a detectable nasal colonization in control untreated animals.

17. The method of claim 16, wherein the step of challenging is conducted less than about 6 months after immunizing.

18. The method of claim 16, wherein the step of challenging is conducted at least 12 hours to less than about 28 days after immunizing.

19. The method of claim 16, wherein the step of challenging is conducted about 7 to 14 days after immunizing.

20. The method of claim 1, wherein the step of challenging comprises administration of the bacteria intranasally.

21. The method of claim 1, wherein the step of challenging comprises administering the selected bacteria in suspension.

22. The method of claim 14, wherein the step of challenging comprises administering from about 10³ CFU to about 10⁹ CFU of S. aureus.

23. The method of claim 1, further comprising identifying an effective vaccine candidate as one that inhibits or prevents S. aureus colonization in the nares of the rodent candidate, or treats or manages existing S. aureus colonization in the nares of in the rodent candidate.

24. The method of claim 1, wherein the step of characterizing the immune response comprises assaying for IgG specific for the vaccine candidate before challenging the rodent candidate with the bacteria.

25. The method of claim 1, wherein the step of characterizing the immune response comprises assaying for IgA specific for the vaccine candidate before challenging the rodent candidate with the bacteria.

26. The method of claim 1, wherein the step of characterizing the immune response comprises evaluating the nasal colonization of bacteria in the rodent candidate after the challenging.

27. The method of claim 1, wherein the step of characterizing the immune response comprises:
assaying the rodent candidate for IgG specific for the vaccine candidate after the immunizing; and
evaluating the nasal colonization of bacteria in the rodent candidate after challenging the rodent candidate with the bacteria.

28. The method of claim 1, wherein the step of characterizing the immune response comprises:
assaying the rodent candidate for IgA specific for the vaccine candidate after the immunizing; and
evaluating the nasal colonization of bacteria in the rodent candidate after challenging the rodent candidate with the bacteria.

29. A rodent model for evaluating the efficacy of a vaccine-candidate for protection against at least one staphylococcal species which comprises:
systemically immunizing a rodent candidate with a Staphylococcus vaccine candidate in an amount sufficient to achieve a detectable immune response; and
detecting the immune response; and
intranasally challenging the rodent candidate with at least one staphylococcal species.

30. The rodent model of claim 29, wherein the rodent candidate is a cotton rat and the at least one staphylococcal species comprises S. aureus.

31. The rodent model of claim 29, which further comprises assaying to determine whether a level of immune response is sufficient to inhibit or prevent colonization of, or to reduce or at least substantially eliminate, staphylococcal colonization in the nares of the rodent candidate.

32. A method for testing vaccine candidates protective against S. aureus which comprises:
subcutaneously immunizing a cotton rat with at least one vaccine candidate;
challenging the cotton rat in or adjacent to the nares with a sufficient amount of S. aureus to allow detection of nasal colonization;
evaluating the immune response in the cotton rat by measuring the level of S. aureus nasal colonization;
wherein a vaccine candidate is selected based on its ability to reduce S. aureus nasal colonization as compared to an appropriate control, to thereby test vaccine candidates protecting against S. aureus.
